(19) United States
(12) Patent Application Publication
(10) Pub. No.: US 2017/0239097 A1
(43) Pub. Date: Aug. 24, 2017

(54) TOTAL CONTACT CAST SYSTEM AND METHODS FOR SAFE AND EASY REMOVAL THEREOF

(71) Applicant: SERENAGROUP, INC., Hingham, PA (US)

(72) Inventors: Thomas E. Serena, Cambridge, MA (US); James Richard Wilcox, Deerfield Beach, FL (US)

(73) Assignee: SERENAGROUP, INC., Hingham, PA (US)

(21) Appl. No.: 15/423,144

(22) Filed: Feb. 2, 2017

Related U.S. Application Data
(60) Provisional application No. 62/290,224, filed on Feb. 2, 2016.

(51) Int. Cl. A61F 13/04; A61F 5/05; A61F 5/01

(52) U.S. Cl. CPC A61F 13/041; A61F 5/0111; A61F 5/05

(57) ABSTRACT

An orthopedic cast system, which includes: a padding layer configured to be disposed within the cast and support a portion of a patient's limb; a casting material configured to harden around the padding layer and form an outer shell of the cast surrounding the patient's limb; and a cutting wire configured to be disposed within the cast and extend along a longitudinal axis of the cast. A total contact cast including a cutting wire between the patient's skin and the casting material forming an outer shell of the cast, as described herein, allows for easy and safe removal of the cast by pulling at least one end of the cutting wire in a direction of the opposite end of the cutting wire so as to cut through the outer shell of the cast.

### CROSS REFERENCE TO RELATED APPLICATIONS

This application claims the benefit of U.S. Provisional Application No. 62/290,224, filed Feb. 2, 2016, the contents of which are incorporated herein by reference in their entirety.

### FIELD OF THE INVENTION

The invention relates to an improved total contact casting system that provides fast, easy, and safe removal of a total contact cast from a patient's body.

### BACKGROUND

Many people suffer from ulcerations, sores, and other types of wounds in one or more appendages, particularly in the feet. For example, people suffering from diabetes also frequently suffer from poor blood circulation in their appendages and, as a result, are at higher risk of developing sores, wounds, or ulcers on their feet. While diabetic foot ulcers account for the majority of neuropathic ulcers on the foot, blockage of arteries supplying blood to the foot, malignancies, venous insufficiency, rheumatoid arthritis and other medical conditions can also cause such ulcers. Due to their location, diabetic and neuropathic foot ulcerations can be quite challenging to heal.

Various types of casts and boots have been developed to treat individuals suffering from diabetic or neuropathic foot wounds. Total Contact Casting was first introduced to the United States over 70 years ago from the leper colonies of India, and has since proven to be particularly efficacious for the treatment of diabetic and neuropathic foot ulcerations. As described by the name, a “total contact cast” is meant to fit the patient's leg and foot very closely, and to touch or be in contact with the patient's whole foot. This intimate contact is designed to secure the foot and leg in a position that allows the patient to ambulate while not allowing the foot to move in the cast, thereby removing pressure from the wound. The initial cast is usually reapplied within 48 to 72 hours after application to accommodate any reduction of swelling and edema in the leg/foot, which is typically followed by a weekly removal and reapplication (changing) of the cast.

The total contact cast typically has a hard outer shell (usually made of plaster or fiberglass), usually applied in several layers, and a soft cast padding (such as gauze, pre-formed cast padding, a stockinet, and/or a compression sock) underneath the outer shell. The purpose of the hard outer shell is to immobilize and protect the patient's particular body part, whereas the purpose of the padding is to provide a comfortable interface between the outer shell and the patient's skin and to protect the patient from injury during removal of the cast by a cast saw. That is during removal of the cast by cutting with a cast saw, it is not uncommon for more pressure than is necessary to be applied to the cast saw, resulting in the oscillating blade penetrating the cast padding and potentially cutting or burning the patient's skin below.

The removal of total contact casting has been a long-standing issue. Prior total contact casting systems had to be spread open at the center while supporting the patient's leg with opposing pressure until the cast broke. This method requires significant strength and can be very time consuming On the other hand, using a cast saw to facilitate cast removal requires operation by a skilled operator and poses the inherent risk of accidental injury to the patient.

A variety of products and approaches have been utilized in attempting to avoid cutting or burning a patient during cast removal, but with little success. As a result, despite the clinical effectiveness of total contact casting for the treatment of neuropathic and diabetic foot ulcerations, conventional removal methods pose a significant problem, resulting in less than 3% of clinicians currently utilizing total contact casting to treat their diabetic and neuropathic foot ulcer patients.

### SUMMARY OF THE INVENTION

It is an object of this invention to overcome the challenges encountered during removal of total contact casts. In particular, it is an object of the invention to provide a means for quicker and easier cast removal, while protecting a patient's skin during the process.

In one aspect of the invention, provided is an orthopedic cast system comprising: a padding layer configured to be disposed within the cast and support a portion of a patient's limb; a casting material configured to harden around the padding layer and form an outer shell of the cast surrounding the patient's limb; and a cutting wire configured to be disposed within the cast and extend along a longitudinal axis of the cast.

In embodiments, the padding layer may be one or more (e.g., multiple) padding layers. The casting material may be a hardening material selected from the group consisting of fiberglass, carbon fiber, thermoplastic, bandaging, casting tape, coated fabrics, and combinations thereof. In certain embodiments, the casting material is configured to begin hardening and form the outer shell upon exposure to air and/or water. A thickness of the outer shell formed by the casting material may range from 1 mm to 4 cm.

The cutting wire of the cast system described herein is preferably flexible, optionally serrated, and configured to cut through the outer shell of the cast when a force pulling at least one end of the wire towards the other end is applied thereto.

In certain embodiments, the cutting wire has: a first end comprising an anchoring member configured to securely anchor the cutting wire in place within the cast; and a second end comprising an attachment portion configured to receive a handle attachment for pulling on the wire. In some embodiments, the second end of the cutting wire may comprise a cap that is removably disposed over the attachment portion. The handle attachment is configured to facilitate pulling on the wire, and may be selected from the group consisting of a ring, a rope, and a molded handle. The anchor piece of the cutting wire may have a flat surface configured to be anchored within or under the casting material. In embodiments, the anchor piece can have any desirable shape selected from the group consisting of circular, oblong, triangular, rectangular, and square.

The cast system described herein may be a total contact cast and configured to receive a portion of the patient's lower leg. Where the cast system is a total contact foot cast, the cast may additionally include a suitable foot base configured to attach to a bottom surface of the cast to allow the patient to walk on the cast.

Embodiments of the invention relate to a total contact cast comprising: a padding layer configured to support a portion of a patient's lower leg; a casting material forming an outer shell of the cast over the padding layer; and a cutting wire between the patient's skin and the casting material, wherein the cutting wire runs parallel to a length of the cast along a longitudinal axis thereof. The cutting wire of the total contact cat preferably has a first end comprising an anchoring member configured to securely anchor the cutting wire within the cast, and a second end comprising an attachment portion configured to receive a handle attachment. The anchoring member of the cutting wire may be securely anchored within or under the casting material.

Embodiments of the invention also relate to methods of applying the total contact cast described herein, including: applying the padding layer to at least a portion of the patient's lower leg; positioning the cutting wire so as to run parallel to the length of the cast along a longitudinal axis thereof; and applying one or more layers of the casting material over the cutting wire and the padding layer. Also provided are methods of removing the total contact cast described herein from a patient's lower leg, including: pulling at least one end of the cutting wire in a direction of the other end so as to cut through the casting material forming the outer shell of the cast.

In further embodiments, the invention relates to methods of treating neuropathic foot wounds/ulcers, including: applying dressing to the foot wound of the patient; covering the lower leg of the patient with a sock; and applying the total contact cast described herein around the patient's lower leg. Such methods may also include pulling at least one end of the cutting wire in a direction of the other end so as to cut through the casting material forming the outer shell of the cast.

Also encompassed by embodiments of the invention is a method of easily and safely removing a total contact cast from a patient's limb, including: providing a cutting wire between the patient's skin and a casting material which forms an outer shell of the cast; and pulling at least one end of the cutting wire in a direction of an opposite end of the cutting wire so as to cut through the casting material disposed over the cutting wire with a cutting surface of the cutting wire. The cutting wire preferably has a first end comprising an anchoring member and a second end comprising an attachment portion configured to receive a handle attachment. The anchoring member can be securely anchored within or under the casting material forming the outer shell of the cast, and the attachment portion may optionally be covered by a cap configured to be removed prior to the attachment portion receiving the handle attachment. In embodiments, the method of removing a total contact cast from a patient's limb may also include: attaching a handle attachment to the attachment portion of the cutting wire; and pulling the cutting wire with the handle attachment so that that the cutting wire cuts through the outer shell of the cast. The cutting wire may be pulled in a distal or proximal direction.

### BRIEF DESCRIPTION OF THE DRAWINGS

FIG. 1 illustrates a cross-sectional view of a representative embodiment of the total contact cast disposed on a patient's leg, wherein the cast is illustrated as being transparent.

FIG. 2 illustrates a cross-sectional view of a representative embodiment of the total contact cast system.

FIG. 3 illustrates the representative total contact cast system during the application process according to an embodiment of the invention.

FIG. 4 illustrates a side view of the representative cutting wire employed in embodiments of the invention.

FIG. 5 illustrates exemplified handle attachments (FIGS. 5A-5D) for the cutting wire employed in embodiments of the invention.

### DETAILED DESCRIPTION OF THE INVENTION

The present disclosure describes for the first time a casting system that is easily and safely removable by medical personnel. Specifically, the casting system comprising a cutting wire and the associated methods described below allow medical personnel to freely cut through the outer shell of the cast during cast removal without the risk of burning or cutting the patient's skin underneath.

The present invention may be understood more readily by reference to the following detailed description of the preferred embodiments of the invention. However, before the casting system, cast, different components and methods are disclosed and described, it is to be understood that this invention is not limited to specific cast types, assemblies or configurations, specific conditions, or specific methods, as such may vary, and any modifications thereto and variations therein will be apparent to those skilled in the art. It is also to be understood that the terminology used herein is for the purpose of describing specific embodiments only and is not intended to be limiting.

It must be noted that as used herein and in the appended claims, the singular forms “a,” “an,” and “the,” include plural forms unless the context clearly indicates otherwise. Thus, for example, reference to “a material” includes one or more of such same or different materials, and reference to “the method” includes reference to equivalent steps and methods known to those of ordinary skill in the art that could be modified or substituted for the methods described herein.

As used herein, “lower leg” may refer to any portion of a patient's leg that is located distal to the patient's knee and including the patient's foot.

As used herein, the terms “patient” and “individual” refer to any person or other subject that can wear or use the described cast system. It is envisioned that the “patient” may also be a non-human animal, such as, e.g., in veterinary applications of the invention. In certain embodiments, the patient is a person suffering from a diabetic or neuropathic ulceration, sore, or other type of wound in an appendage (e.g., lower leg).

### Casting System

It will be appreciated that, while the referenced cast is described herein for use as a total contact cast for treatment of ulcerations, sores, and/or any other wounds on a foot, the cast can easily be modified to be used for any other suitable purpose. For example, it is envisioned that the cast can be used for protecting an appendage with a broken and/or fractured bone while the bone heals, and/or for immobilizing or restricting access to an appendage. It is furthermore envisioned that the cast can be configured to, e.g., extend up a patient's leg and past the knee. Accordingly, in certain embodiments of the invention, the cast (not shown) is configured to extend over one or more appendages of a patient, such as the patient's arm, finger, and/or torso. In such embodiments, the cast may be configured to, e.g., extend over a posterior portion of a patient's forearm, wrap over a distal end of the patient's hand (e.g., over the distal end of the patient's fingers and/or with one or more of the patient's fingers extending through a corresponding hole or holes in the casting material), or extend over an anterior portion of the patient's forearm.

The various components of the total contact cast system described herein can be used in any suitable manner that allows the components (individually or in combination) to help protect a wounded portion of a patient's limb, while allowing for easy and safe removal of the cast when necessary. The various components of the described cast system can be used in any suitable combination, meaning that portions of the cast system can be omitted and/or replaced with one or more known or novel components, and any additional components (such as, e.g., wound dressing, casting material, boot, footplate, shoe, ointment, etc.) can be added or used with one or more components of the described cast system.

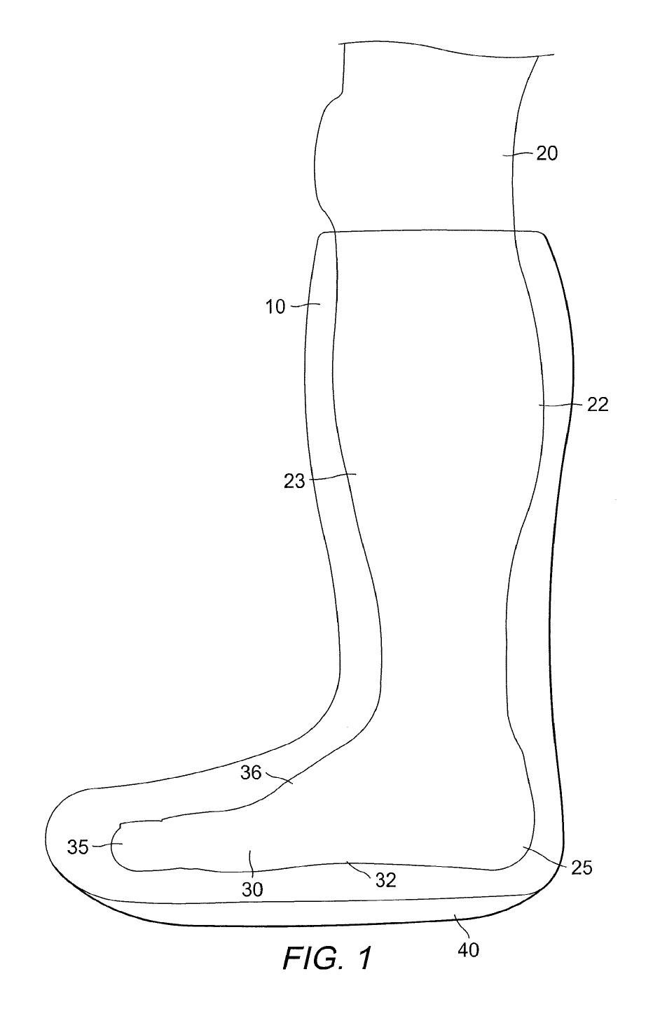

FIG. 1 shows a patient's lower leg (20), covered with a cast (10). The cast (10) may be configured so as to extend over a first surface of a patient's appendage (e.g., lower leg), over an end of the appendage (e.g., foot), and over a second surface of the appendage, which is substantially opposite to the first surface. For example, the cast (10) may be configured so as to extend: over a posterior portion (22) of a patient's lower leg (20) (e.g., calf), over the patient's heel (25), across a length of the sole (32) of the patient's foot (30), over an anterior portion (35) of the foot (30) (e.g., toes), across a dorsal portion (36) of the foot (30); and over an anterior portion (23) of the lower leg (20) (e.g., shin).

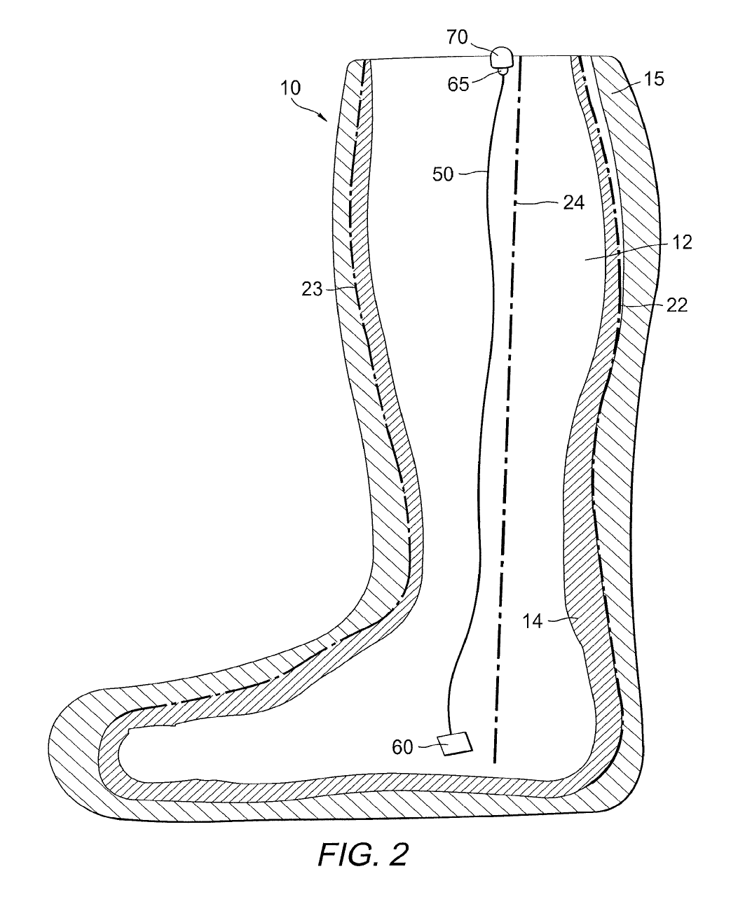

As shown in FIG. 2, the cast (10) may be an orthopedic foot cast (e.g., total contact cast). The cast (10) may comprise a padding layer (14) positioned between the patient's skin (optionally covered by a compression sock (12)) and the casting material (15).

### Casting Material

The cast (10) described herein can comprise any suitable casting material (15) that allows the cast (10) to substantially conform to the shape of a patient's appendage (e.g., lower leg (20)) during application and then harden in that shape to form an orthopedic cast.

Examples of suitable casting materials (15) include (but are not limited to) any known or novel: fiberglass casting material (e.g., fiberglass that has been impregnated with polyurethane and/or another suitable material, fiberglass that is coated with and/or otherwise comprises silver, copper, nickel, iodine, bamboo, and/or another suitable antimicrobial material and/or anti-odor material, and/or combinations thereof); hardenable bandaging (e.g., cotton bandaging that is coated with plaster, polyester bandaging that is covered with plaster, and/or any other suitable bandaging material that is configured to harden after application); a thermoplastic casting material; a polypropylene casting material; a carbon fiber casting material; a hardenable antimicrobial material, such as, e.g., a plaster or another suitable hardenable material covering a bamboo cloth, a bamboo rayon, a polyester made from bamboo, other types of fabric comprising bamboo, and/or a silver-coated fabric (e.g., silver coated polyethylene terephthalate, silver-coated rayon, silver-coated polyester, etc.), a copper-coated fabric, a silver/copper-coated fabric, a silver/copper/nickel-coated fabric, a silver/copper/tin-coated fabric, an iodine incorporated cloth, and/or any other suitable material having one or more antimicrobial and/or anti-odor features; a casting material (e.g., fiberglass, cotton bandaging, and/or any other suitable casting material) that is impregnated with and/or otherwise comprises a suitable antibiotic, bactericide, and/or other antimicrobial ingredient (e.g., penicillin, amikacin, gentamicin, kanamycin, netimicin, tobramycin, streptomycin, spectinomycin, geldanamycin, herbimycin, rifaximin, ertapenem, doripenem, imipenem, meropenem, defadroxil, cefazolin, cefalotin, cephalexin, and/or any other suitable antimicrobial); casting tape; and/or any other suitable material that is configured to be formed around a portion of a patient's limb and then hardened to form an orthopedic cast.

In certain embodiments, the outer shell is of a casting material (15) (e.g., fiberglass) that is configured to begin hardening once it is exposed to air and/or water.

The cast (10) can comprise any suitable number of layers of casting material (15). For example, the cast (10) of the invention may comprise between 1 and 20 layers of casting material (15), including any suitable number of layers of casting material (15) that fall within the aforementioned range, such as, e.g., between 5 and 10 layers. The casting material (15) can also have any suitable thickness, such as between 1 mm and about 4 cm.

In embodiments, the casting material (15) may be in the form of a plurality of strips or a single strip configured to form an outer shell of the cast (10) on an appendage (e.g., lower leg). The strips can have any suitable shape, such as, e.g., rectangular, belt-shaped, triangular, rounded, polygonal, irregular, symmetrical, asymmetrical, ribbon-like, cord-like, rope-like, and/or any other suitable shape. The casting material (15) may furthermore be of any desired length or lengths (if a plurality of strips are to be applied) to form an outer shell of the cast (10) of the desired thickness and covering the desired body part. For example, some of the strips may be configured to circumscribe a portion of the cast, some of the strips may be configured to extend only partially around a part of the cast, or some of the strips may be configured to overlap with each other. While the strips can run substantially perpendicular to a length (or a longitudinal axis) of the cast (10), the straps may also, or alternatively, be configured to run at an angle with respect to the length (or a longitudinal axis) of the cast (10).

### Padding Layer

With respect to the padding layer (14), the cast (10) of the invention may include any suitable amount or layers of padding in any suitable location (e.g., over a patient's toes, dorsal portion of the patient's foot, shin, calf, ankle, a lateral side of a person's lower leg, or any other body part to be cast that could benefit from additional padding. The aforementioned padding (14) can be modified in any suitable manner that allows it to pad, provide shear reduction to, ventilate, and/or otherwise protect an appendage within a cast (e.g., cast (10)). In certain embodiments, additional support, protection, and/or padding may be provided in the form of a sock, such as a compression sock (12).

Where the cast (10) comprises a padding layer (14), any suitable type of padding material may be used, including (but not limited to) closed cell padding, open cell padding, polymer padding (e.g., polypropylene, polyethylene, polyurethane, etc.), foam, silicon, gel, cotton, vinyl, polyvinyl chloride, ethylene vinyl acetate, cork, and/or combinations thereof. Although some embodiments of the cast system (10) comprise a single type or layer of padding material (14), in other embodiments, the cast system (10) comprises a plurality of types and/or layers of padding material (14).

In certain embodiments, the padding layer (14) may be attached to a portion of any suitable type of sock (e.g., a compression sock (12)) or other material directly disposed over the patient's body part (e.g., lower leg, hand, arm, etc.). The padding layer (14) may be configured and applied to the patient's body part so as to move completely independently of the outer shell of the cast formed by the casting material (15). Alternatively, one or more of the padding layers (14) may be at least partially attached to the casting material (15) when the cast (10) is disposed on the body part. As a result of the latter embodiment, the described padding layer (14) is able to reduce shearing forces between the casting material (15) and the patient's skin.

### Cutting Wire

As additionally shown in FIG. 2, the cast (10) of the invention includes a cutting wire (50) that is positioned within the cast (10) between the patient's skin (optionally covered by a compression sock (12)) and the casting material (15), such as, e.g., between the padding layer (14) and the casting material (15). As shown by the marked lines in FIG. 2, the cutting wire (50) may be positioned along a longitudinal axis of the cast (10), e.g., along a central portion (24) of the cast (10), along a posterior side (22) of the cast (10), or along an anterior side (23) of the cast (10).

An exemplary embodiment of the aforementioned cutting wire (50) is shown in FIG. 4. The cutting wire (50) preferably has a first end comprising an anchoring member (60) for securely anchoring the cutting wire (50) in place within the cast, and a second end comprising an attachment portion (65) for attaching a handle attachment (not shown) to facilitate pulling on the wire during removal of the cast.

In certain embodiments, the attachment portion (65) of the cutting wire (50) may comprise/be covered with a removable cap (70) that is removed by medical personnel prior to attaching/securing a handle attachment (exemplary handle attachments shown in FIGS. 5A-5D) to the attachment portion (65). The removable cap (70) that covers the attachment portion (65) of the cutting wire (50) may be constructed of a material having a contrasting color so as to stand out from the casting materials of the cast. For example, the removable cap (70) may be of a blue, red, yellow, pink, or other uniquely colored material so as to be easily distinguished from the conventionally white or off-white materials of the padding layer and/or the casting material. In this manner, the casting technician or medical personnel can readily identify the exact location of the cutting wire (50) within the cast during cast removal.

In embodiments, the attachment portion (65) of the cutting wire (50) is configured to receive a handle attachment to facilitate cast removal by medical personnel by pulling the cutting wire (50) using the handle attachment. In some embodiments, the cutting wire (50) may include an attachment portion at both ends of the wire configured to receive handle attachments. In such embodiments (not shown) two handle attachments may be attached (one to each end of the wire) to facilitate cast removal by medical personnel by pulling the ends of the cutting wire (50) towards each other.

Examples of suitable handle attachments for the cutting wire (50) are shown in FIGS. 5A-5D, and include (but are not limited to): a ring (72) that may optionally include a separate coupling (e.g., screw-in) portion (73) to facilitate attachment to the attachment portion (65); a chord (74), such as a parachute chord that may be attached/tied to the attachment portion (65); or a molded handle (75) having an attachment base (76) or attachment rope (77) that may hook or attach to the attachment portion (65).

The cutting wire (50) used in embodiments of the invention may be any conventional or novel cutting wire, blade, or wire saw, including (but not limited to) spiral, serrated, sabercut and flat wires/blades suitable for cutting through the cast in which it is employed. A spiral wire/blade allows cutting in any direction due to all edges being cutting edges. The cutting wire (50) may be of any suitable material, including, but not limited to, metals (solid or tipped), such as steel, hardened steel, stainless steel, titanium, titanium alloys, tungsten carbide, brass, and/or cobalt, or even plastic materials suitable for cutting through the outer shell of the cast (10). The cutting wire (50) may be of a length substantially corresponding to the longitudinal length of the cast in which it is employed. Moreover, the cutting wire (50) is preferably positioned so as to run along a longitudinal axis of the cast in which it employed.

### Associated Methods

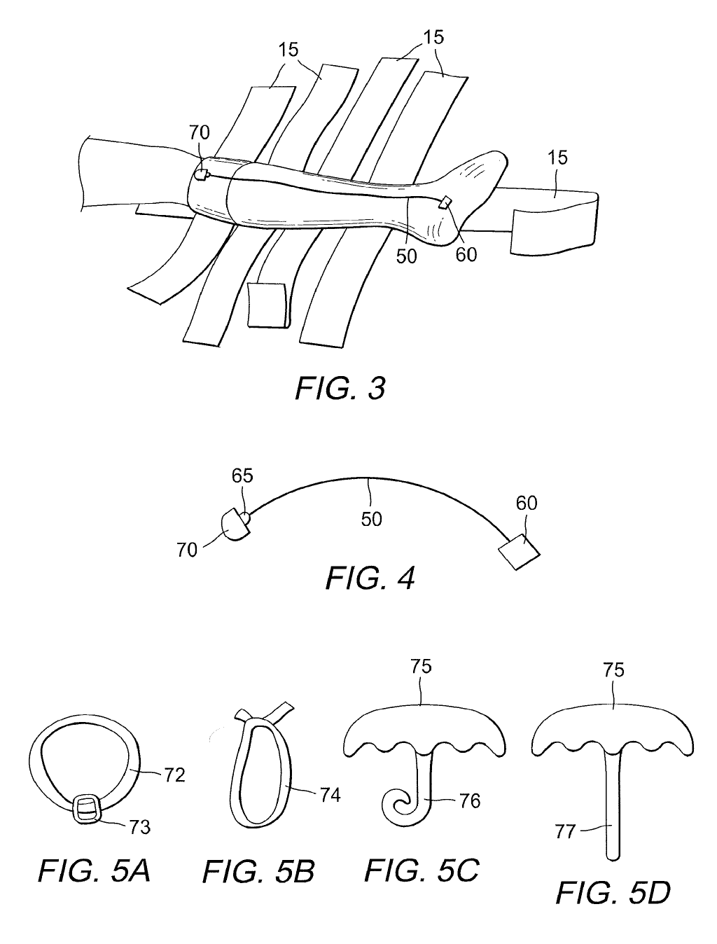

Although the described components of the cast system (10) can be applied to a patient in any suitable manner, FIG. 3 shows an embodiment of a method of application of the casting material (15) and the cutting wire (50) to a patient's lower leg. This application method may, however, be modified in any suitable manner (including by rearranging, adding to, removing, substituting, and/or otherwise modifying one or more steps of the method).

As shown in FIG. 3, embodiments of the method include applying the casting material (15) to a patient's limb (e.g., lower leg). The casting material (15) is preferably applied over a padding layer (not shown) and/or a compression sock that covers the patient's skin. In embodiments, the method furthermore includes providing the cutting wire (50) under the casting material (15). In the exemplary embodiment depicted in FIG. 3, the cutting wire (50) may be positioned to run parallel to a longitudinal axis of the lower leg, with a first end of the cutting wire (50) comprising an anchoring member (60) that becomes securely anchored/adhered under or within the casting material (15) upon hardening, and a second end of the cutting wire (50) comprising an attachment member (70) to which a separate handle attachment may be attached by medical personnel during cast removal. Also encompassed by the method are embodiments wherein the first and second ends of the cutting wire (50) are positioned in the reverse direction, i.e., the anchoring member being positioned at the upper portion of the cast (near the upper portion of the patient's lower leg) and the attachment portion being positioned at the lower portion of the cast (near the foot of the patient).

In one aspect, the present invention provides a method that includes inserting a cutting wire as described herein between the patient and the orthopedic cast during application of the casting material to the patient's limb, wherein at least a portion of the cutting wire becomes securely adhered to or embedded within the cast and at least one end of the cutting is positioned so as to be pulled by a user (medical personnel) during cast removal. As described herein with reference to FIG. 4, the cutting wire (50) may initially comprise a cap (70) over the attachment portion (65) of the cutting wire (50) that is removed by the user (medical personnel) prior to attaching a handle attachment to the attachment portion (65) at the time of cast removal.

Methods encompassed by the invention may additionally include applying any suitable and known or novel wound dressing to a wound prior to application of a padding layer and/or orthopedic sock and the casting material (15).

The casting material (15) can be applied in the form of multiple elongated strips as shown or as a single strip (not shown). Suitable application methods include those known in the art, and may include the application of multiple layers of casting material so as to form an outer shell of the cast having the desired stability, support, hardness, and/or thickness.

By providing the cutting wire (50) within the aforementioned cast system (10), embodiments of the invention provide for methods of easily and safely removing a cast from the patient's limb (e.g., lower leg).

In one aspect, the present invention provides a method of cutting an orthopedic cast by pulling on the cutting wire embedded in or under the hardened outer shell of the cast in a distal or proximal direction (at the discretion of the user or medical personnel), thereby cutting through the casting material forming the hardened outer shell without the need for scissors or oscillating cutting saws.

Among the advantages provided by the described cast system is the ability to cut through the casting material of the cast by pulling on the cutting wire. Specifically, the cutting if the cast during cast removal is performed without the need for scissors or use of a cast saw.

Also intended within the scope of the invention are methods of treatment of an appendage by various embodiments of the referenced cast system.

It should also be understood that the foregoing relates to preferred embodiments of the present invention and that numerous changes may be made therein without departing from the scope of the invention. The invention is further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present invention and/or the scope of the appended claims.

What is claimed is:

1. An orthopedic cast system, comprising:
a padding layer configured to be disposed within the cast and support a portion of a patient's limb;
a casting material configured to harden around the padding layer and form an outer shell of the cast surrounding the patient's limb; and
a cutting wire configured to be disposed within the cast and extend along a longitudinal axis of the cast.

2. The cast system according to claim 1, wherein the casting material is a hardening material selected from the group consisting of fiberglass, carbon fiber, thermoplastic, bandaging, casting tape, coated fabrics, and combinations thereof.

3. The cast system according to claim 1, wherein the casting material is a hardening material that is configured to begin hardening and form the outer shell upon exposure to air and/or water.

4. The cast system according to claim 1, wherein a thickness of the outer shell formed by the casting material is between 1 mm and 4 cm.

5. The cast system according to claim 1, wherein the cutting wire is flexible, optionally serrated, and configured to cut through the outer shell of the cast when a force pulling at least one end of the wire towards the other end is applied thereto.

6. The cast system according to claim 1, wherein the cutting wire has:
a first end comprising an anchoring member configured to securely anchor the cutting wire in place within the cast; and
a second end comprising an attachment portion configured to receive a handle attachment for pulling on the wire.

7. The cast system according to claim 6, wherein the second end further comprises a cap that is removably disposed over the attachment portion.

8. The cast system according to claim 6, wherein the handle attachment is configured to facilitate pulling on the wire and is selected from the group consisting of a ring, a rope, and a molded handle.

9. The cast system according to claim 6, wherein the anchor piece has a flat surface configured to be anchored within or under the casting material.

10. The cast system according to claim 6, wherein the anchor piece has a shape selected from the group consisting of circular, oblong, triangular, rectangular, and square.

11. The cast system according to claim 1, wherein the cast is a total contact cast configured to receive a portion of the patient's lower leg.

12. The cast system according to claim 11, further comprising a foot base configured to attach to a bottom surface of the cast to allow the patient to walk on the cast.

13. A total contact cast, comprising:
a padding layer configured to support a portion of a patient's lower leg;
a casting material forming an outer shell of the cast over the padding layer; and
a cutting wire between the patient's skin and the casting material, wherein the cutting wire runs parallel to a length of the cast along a longitudinal axis thereof.

14. The total contact cast according to claim 13, wherein the cutting wire has a first end comprising an anchoring member configured to securely anchor the wire within the cast, and a second end comprising an attachment portion configured to receive a handle attachment.

15. The total contact cast according to claim 14, wherein the anchoring member is securely anchored within the casting material.

16. The total contact cast according to claim 14, wherein the anchoring member is securely anchored under the casting material.

17. The total contact cast according to claim 13, wherein the casting material is a hardening material selected from the group consisting of fiberglass, carbon fiber, thermoplastic, bandaging, casting tape, coated fabrics, and combinations thereof.

18. The total contact cast according to claim 13, further comprising a foot base along a bottom surface of the cast.

19. A method of applying the total contact cast of claim 13 to a patient's lower leg, comprising:
applying the padding layer to at least a portion of the patient's lower leg;
positioning the cutting wire so as to run parallel to the length of the cast along a longitudinal axis thereof; and
applying one or more layers of the casting material over the cutting wire and the padding layer.

20. A method of removing the total contact cast of claim 13 from a patient's lower leg, comprising:
pulling at least one end of the cutting wire in a direction of the other end so as to cut through the casting material forming the outer shell of the cast.

21. A method of removing a total contact cast from a patient's limb, comprising:
providing a cutting wire between the patient's skin and a casting material which forms an outer shell of the cast; and
pulling at least one end of the cutting wire in a direction of an opposite end of the cutting wire so as to cut through the casting material disposed over the cutting wire with a cutting surface of the cutting wire.

22. The method of claim 21, wherein:
the cutting wire has a first end comprising an anchoring member and a second end comprising an attachment portion configured to receive a handle attachment, the anchoring member being securely anchored within or under the casting material forming the outer shell of the cast;
the attachment portion may optionally be covered by a cap configured to be removed prior to the attachment portion receiving the handle attachment.

23. The method of claim 22, comprising:
attaching a handle attachment to the attachment portion of the cutting wire; and
pulling the cutting wire with the handle attachment so that that the cutting wire cuts through the outer shell of the cast.

24. The method of claim 23, wherein the pulling of the cutting wire is in a distal or proximal direction.
